(19) United States
(12) Patent Application Publication
(10) Pub. No.: US 2010/0047195 A1
(43) Pub. Date: Feb. 25, 2010

(54) LIP BALM AND METHOD OF TREATING LIP CONDITIONS

(76) Inventor: David Elbaum, Bakersfield, CA (US)

Correspondence Address: FULBRIGHT & JAWORSKI L.L.P., 600 CONGRESS AVE., SUITE 2400, AUSTIN, TX, 78701, US

(21) Appl. No.: 12/303,492

(22) PCT Filed: May 16, 2008
(86) PCT No.: PCT/US08/64018

§ 371 Date: Jun. 17, 2009

(30) Foreign Application Priority Data
May 18, 2007 (US) 60939001

(51) Int. Cl. A61K 8/18; A61K 31/56; A61K 31/58; A61P 17/00; A61Q 17/04

(52) U.S. Cl. 424/59; 514/169; 514/174; 514/178

(57) ABSTRACT

A pharmaceutical composition for treating a lip condition. The pharmaceutical composition includes a therapeutically effective amount of a corticosteroid, or a physiologically acceptable salt of the corticosteroid, and a physiologically acceptable carrier In the composition, the corticosteroid does not include hydrocortisone. Also provided is a method of using the composition to treat a lip condition such as inflammation or infection.

### REFERENCE TO PRIOR APPLICATIONS

This application claims the benefit of U.S. Provisional Application No. 60/939,001, filed on May 18, 2007, which is incorporated by reference herein.

### BACKGROUND

1. Field of Disclosure

This disclosure relates generally to compositions, including lip balms, for treating lip conditions.

2. Background

It is estimated that over 90% of patients treated with oral isotretinoin (13-cis-retinoic acid) develop cheilitis (cracking, soreness and other inflammation of the lips). Such patients are not only subject to lip pain, but the lesions can also become infected, causing additional health problems. The present invention provides a lip balm for treating and preventing cheilitis, infection, and other conditions of the lip. Also provided are other compositions such as creams and solutions for treating lip conditions. The lip balm and other compositions can provide for less swelling, less pain, better quality of life, and make it easier to complete a course of isotretinoin treatment.

### SUMMARY

In one aspect, a pharmaceutical composition for treating a lip condition is provided. The composition includes: a) a therapeutically effective amount of a corticosteroid, or a physiologically acceptable salt thereof, and b) a physiologically acceptable carrier. A proviso is included in that the corticosteroid does not include the particular corticosteroid hydrocortisone.

In another aspect, a method of treating a lip condition in a subject in need of such treatment is provided. The method includes applying a pharmaceutical composition to one or both lips of a subject under conditions sufficient to treat the lip condition. The pharmaceutical composition includes a) a therapeutically effective amount of a corticosteroid, or a physiologically acceptable salt thereof; and b) a physiologically acceptable carrier, with the proviso that the corticosteroid does not include hydrocortisone.

The foregoing has outlined rather broadly the features and technical advantages of the present disclosure in order that the detailed description that follows may be better understood. Additional features and advantages will be described hereinafter which form the subject of the claims. It should be appreciated by those skilled in the art that the conception and specific configurations disclosed may be readily utilized as a basis for modifying or designing other structures for carrying out the same purposes of the present disclosure. It should also be realized by those skilled in the art that such equivalent constructions do not depart from the spirit and scope of the invention as set forth in the appended claims. The novel features which are believed to be characteristic of the disclosure, both as to its organization and method of operation, together with further objects and advantages will be better understood from the following description.

### DETAILED DESCRIPTION

In one version of the pharmaceutical composition, a lip balm comprises the following components, with amounts in % by weight (or wt. %):

| Component | wt. % |
|---|---|
| triamcinolone acetonide | 0.1 |
| silica gel micronized | 0.5 |
| wax, white beadlets | 25 |
| petrolatum USP white | 64 |
| potassium sorbate | 0.1 |
| butylated hydrotoluene (BHT) | 0.1 |
| stevia USP | 0.2 |
| mineral oil | 9 |
| flavors (bubble gum/marshmallow) | varies |

The total wt. % can be brought to 100% by inclusion of a carrier, lubricant, solvent, or other component.

To prepare the lip balm, the wax and petrolatum are melted together. Silica gel, steroid, potassium sorbate, BHT, and stevia (all in powder form) are triturated together, then wet with mineral oil. The wetted substances are then added to the melted wax and petrolatum, and the combination mixed until uniform. Flavors are added at this point, with continued mixing. Finally, the mixture is poured into lip balm tubes.

Various ingredients are available from commercial sources. For example, triamcinolone acetonide can be obtained from Spectrum (Tucson, Ariz.). The silica gel, wax, petrolatum, BHT, mineral oil and flavors can be obtained from Professional Compounding Centers of America (Houston, Tex.). The potassium sorbate and stevia can be obtained from Medisca, (Plattsburgh, N.Y.).

Triameinolone acetonide is a corticosteroid for use as an anti-inflammatory agent. In general, any corticosteroid (or its physiologically or pharmaceutically acceptable salt) can be used in the composition as long as it has an effect in reducing the extent of inflammation. In preferred embodiments, the corticosteroid does not include hydrocortisone. In other embodiments, the corticosteroid does not include hydrocortisone or physiologically acceptable salts thereof. Examples of other corticoidsteriods that can be used include, but are not limited to, mometasone, hydrocortisone acetate, betamethasone valerate, fluocinolone acetonide, flupamesone, dexamethasone, hydrocortisone acetate, hydroxy hydrocortisone, hydrocortisone valerate, triamcinolone hexacetonide, amcinonide, fluocinolone acetonide, fluocinonide, flurandrenolide, diflurrasone diacetate, betamethasone dipropionate, betamethasone, betamethasone benzoate, betamethasone valerate, halcinonide, desoximethasone, desonide, prednisolone, clocortolone pivalate, physiologically acceptable salts thereof, or mixtures thereof. Depending on the corticosteroid, the amount of steroid can preferably range from about 0.05 wt. % to about 5 wt. % of the lip balm composition, and preferably from about 0.1 wt. % to about 1 wt. %. For example, mometasone can be used at about 0.1 wt. %. Table 1 lists steroid potency ranking and different types of steroids that can be used in connection with a lip balm.

TABLE 1
Topical Steroid Potency Ranking
These commonly used topical steroids are listed by potency
group, from Group I as the super-potent category to Group VII
is the least potent. There is no significant difference between
agents within Groups II through VII (the compounds are listed
alphabetically). In Group I, Temovane and Ultravane
are more potent than the other.

| Group | Brand Name | Generic Name |
|---|---|---|
| | Temovate cream 0.05% | clobetasol proprionate |
| | Temovate ointment 0.05% | |
| | Ultravate cream 0.05% | halobetasol proprionate |
| | Ultravate ointment 0.05% | |
| | Diprolene ointment 0.05% | betamethasone dipropionate |
| | Psorcon ointment 0.05% | difforasone diacetate |
| Group II | Cyclocort ointment 0.1% | amcinonide |
| | Diprolene AF cream | betamethasone dipropionate |
| | Elocon ointment 0.1% | mometasone furoate |
| Group | Aristocort A ointment 0.1% | triamcinolone acetonode |
| III | Cutivate ointment 0.005% | fluricasone propionate |
| | Cyclocort cream 0.1% | amcinonide |
| | Cyclocort lotion 0.1% | |
| Group | Elocon cream 0.1% | mometasone furoate |
| IV | Elocon lotion 0.1% | |
| | Synalar ointment 0.025% | fluocinolone acetonide |
| | Westcort ointment 0.2% | hydrocortisone valerate |
| Group V | Cordran cream 0.05% | flurandrenolide |
| | Cutivate cream 0.05% | fluricasone propionate |
| | Locoid Lipocream 0.1% | hydrocortisone butyrate |
| | Locoid cream 0.1% | hydrocortisone butyrate |
| | Locoid ointment 0.1% | |
| | Locoid solution 0.1% | |
| | Synalar cream 0.025% | fluocinolone acetonide |
| | Westcort cream 0.2% | hydrocortisone valerate |
| Group | Aclovate cream 0.05% | alclometasone dipropionate |
| VI | Aclovate ointment 0.05% | |
| | Synalar cream 0.01% | fluocinolone acetonide |
| | Synalar solution 0.01% | |
| | Desowen cream 0.05% | desonide |
| | Desowen lotion 0.05% | |
| Group | Pramosone cream 1.0% | hydrocortisone acetate |
| VI | Premosone cream 2.5% | pramoxine hydrochloride 1% |
| | Premosone lotion 1.0% | |
| | Premosone lotion 2.5% | |
| | Premosone ointment 1.0% | |
| | Premesone ointment 2.5% | |
| | Hytone cream 2.5% | hydrocortisone |
| | Hytone lotion 2.5% | |
| | Hytone ointment 2.5% | |
| | Others containing | |
| | dexamethasone, | |
| | fluromethalone, prednisolone, | |
| | and methylprednisolone | |

In certain versions, so called “natural” anti-inflammatory agents can be used. Such agents include, but are not limited to, candelilla wax, alpha bisabolol, aloe vera, Manjistha (extracted from plants in the genus Rubia, particularly Rubia cordifolia), Guggal (extracted from plants in the genus Commiphora, particularly Commiphora mukul, and curcumin (extracted from tumeric).

Silica gel is used as a suspending agent. Examples of other suspending agents that can be used include, but are not limited, to silica, magnesium silicate, aluminum silicate, veegum, kaolin, and mixtures thereof.

Wax and petrolatum act as carriers or vehicles for the active ingredients of the lip balm. Other physiologically or pharmaceutically acceptable carriers well known in the art can be used. Such carriers are described in Remington's Pharmaceutical Sciences, 17th edition, 1985, ed. Alfonso R. Gennaro, Mack Publishing Company, Easton, Pa. 18042. Examples of other useful waxes include, but are not limited to, stearyl alcohol, myristal alcohol, cetyl alcohol, tridecyl alcohol, microcrystalline, lanolin, paraffin, ozokerite, lanolin alcohol, hydrogenated lanolin, candelilla, cocoa butter, petrolatum, shellac, hydrogenated castor oil, spermaceti, bran wax, capok, bayberry, and mixtures thereof.

Examples of other carriers include, but are not limited to, water and aqueous systems; glycerin; various hydrophilic solvents including alcohols such as ethanol, methanol, propyl and other alcohols; or any combinations thereof. In addition, the vehicle of the compositions according to the present invention can be in the form of a suspension, solution, mixture, homogeneous phase formulation or in the form of an emulsion, including, but not limited to, oil-in-water, water-in-oil and multiple phase emulsions. These emulsions can cover a broad range of consistencies including a thin lotion, creamy lotion, light cream, and heavy cream. Other suitable topical carriers include an anhydrous liquid solvent such as oil and alcohol; aqueous-based single phase liquid solvent (e.g. hydro-alcoholic solvent system); anhydrous solid and semisolid (such as a gel and a stick); and aqueous based gel system.

Potassium sorbate is included as a preservative. Other preservatives that can be used include, but are not limited to, sodium benzoate, methyl paraben, propyl paraben, and mixtures thereof.

Butylated hydroxytoluene (BHT) is an anti-oxidant. Other anti-oxidants that can be used include, but are not limited to, butylated hydroxyanisole (BHA), d,l-alpha tocopherol (Vitamin E), and mixtures thereof.

Stevia is a naturally occurring sweetener extracted from stevia leaves. In general, any natural or artificial sweetener can be used. Other examples include, but are not limited to, dipeptide sweeteners, amino acid-based sweeteners, sucrose, fructose, glucose (corn syrup), dextrose, invert sugar; saccharine and its salts; cyclamic acid and its salts; aspartame; dihydrochalcone; glycyrrhizin; sorbitol, mannitol, xylitol; hydrogenated starch hydrolysate, 3,6-dihydro-6-rnethyl-1-1,2,3-oxathiazin-4-one-2,2-dioxide (acesulfame-) and its salts; talin; sucralose; and mixtures thereof.

Mineral oil acts as a lubricant. Other lubricants can be used, including but not limited to lanolin, derivatives of lanolin such as the ethoxylated acetylated alcohol and surface active alcohol derivatives of lanolin, propylene glycol, polypropylene glycol, polyethylene glycol, lanolin and lanolin derivatives, other mineral oils, fatty alcohols, glycerin, and mixtures thereof.

Other flavors, well known in the art, can be used in preparing compositions.

As noted above, the wt. % of steroid can range from about 0.05 wt. % to about 5 wt. %, depending on the particular steroid. The wt. % of other components of the lip balm can vary to adjust for the particular medical use and form (for example, gel, cream, lotion) of the composition. For example, the sweetness, flavors, consistency, and/or amount of preservative can be adjusted. In addition, the inclusion of other components such as sunscreen (see below) may require adjusting the wt. % of the coinmponents depending on the medical use and form of the composition.

As used herein, the tern “lip balm” refers to a solid or semi-solid composition for applying to the lips. In other embodiments, a pharmaceutical composition can be modified to prepare, for example, a cream by changing the wax to a cream base. Thus, the form of different versions of the pharmaceutical composition can be a cream, emulsion, gel, liquid, lotion, mousse, patch, pomade, powder, solid, spray, stick or towelette. Also, a composition may optionally include one or more of the following additional ingredients: anesthetics (such as pramoxine), anti-allergenics, antifungals, antiseptics, antibacterials, antivirals (such as acyclovir), depigmenting agents, emollients, exfollients, fragrances, humectants, pharmaceutical agents, preservatives, skin protectants, skin penetration enhancers, stabilizers, surfactants, thickeners, viscosity modifiers, vitamins, and mixtures thereof.

For example, in various embodiments, a pharmaceutical composition may have one or more sunscreen actives. Sunscreen actives that can be used include those for UVA and UVB protection (290 to 400 nanometer solar radiation). The sunscreen active may be any organic or inorganic compound known in the art, including but not limited to, oxybenzone, sulisobenzone, dioxybenzone, menthyl anthranilate, para aminobenzoic acid (PABA), octyl methoxycinnamate, octocrylene, DEA methoxycinnamate, drometrizole trisiloxane, octyl salicylate, homomenthyl salicylate, octyl dimethyl PABA, TEA salicylate, titanium dioxide, zinc oxide, butylmethoxy dibenzoylmethane (avobenzone), 4-methyl benzilidene camphor, octyl triazone, terephthalydiene dicamphor sulfonic acid, ethyl PABA, hydroxy methylphenyl benzotriazole, methylene bis-benzotriazoyltetramethylbutylphenol, bis-ethylhexyloxyphenol methoxyphenol triazine, and mixtures of the foregoing.

Typically, sunscreen actives can range from about 0.1 wt % to about 80 wt %, and more preferably about 1 wt % to about 30 wt %, based on the total weight of the composition. The present composition can be formulated to deliver from about 2 to about 70 SPF in sunscreen protection, more preferably about 15 to about 30 SPF.

For compositions that contain an antiviral agent, examples of antiviral agents that can be used include, but are not limited to, acyclovir, benzalkonium chloride, alcohol, allantoin, anhydrous glycerin, benzocaine, camphor, carbamide peroxide, lanolin, menthol, petrolatum, phenol, calcium pantothenate, lactic acid, salicylic acid, cantharidin, podophyllin, urea, idoxuridine, amantadine, methisazone, cytarabine, interferons, chloroform, ether, bacillus calmette-guerin, levamisole, and mixtures thereof.

For compositions that contain an antifungal agent, examples of antifungal agents that can be used include, but are not limited to, haloprogin, iodochloro, miconazole nitrate, tolnaftate, thiabendazole, chloroxine, amphotericin, candicin, fungimycin, nystatin, chlordantoin, clotrimazole, ethonam nitrate, miconazole nitrate, pyrrolnitrin, fezatione, ticlatone, tolnaftate, triacetin, carbonic acid derivatives; dithiocarbamate, thiourea, thiocyantes; aromatic carboxylic acids and the amides thereof, benzoic acid, salicylic acid, salicylic acid amide and anilide; aromatic sulfides, polysulfides, and sulfoxides, 5,5-dichloro-2,2-dihydroxydiphenylsulfide; invert soaps, quaternary ammonia and phosphonium compounds, decamethylene-bis-(4-thio-pyridine-methyl-tosylate; quinoline derivatives, 8-hydroxyquinoline sulfate, halogenated quinolines, 7-iodo-8-hydroxy-quinoline-5-sulfonic acid, 5-chloro-7-iodo-8-hydroxy-quinoline, 5-chloro-8-hydroxy-quinoline, 5,7-dichloro-8-hydroxyquinaldine, 5,7-di iodo-8-hydroxyquinoline, decamethylene-bis (4-am ino-qu inaldium chloride); benzothiazole derivates, (2-dimethylamino-6-(beta-diaminoethoxy)-benzothiazole dihydrochloride; imidazole derivatives, 1-(o-chloro-alpha-alpha-diphenyl-benzyl)-imidazole, 1-[o,p-dichloro-beta-(o,p-dichlorobenzyloxy)-phenethylimidazole]; benzimidazole derivatives, 2-phenylbenzimidazole, 2-furfurylbenzimidazole; thiadizine derivatives, 3,5-dibenzyltetrahydro-1,3,5-thiadizine-2-thione; furan derivatives, 5-nitro-2-furfuryl-3-chloropropionate; quinones, tetrachloro-p-benzoquinone, 1,4-naphthoquinone, phenanthraquinone; sulfonamides and sulfones; aromatic diamidines, 2-hydroxystilbamidine, diamidinodiphenylamine, and mixtures thereof.

For compositions that contain an antibacterial agent, which can be an antibiotic agent or an antibacteriostatic agent, or include both, examples of antibacterial agents that can be used include, but are not limited to, honey, sulfonomides, penicillins, cephalosporins, penicillinase, lincomycins, vancomycins, tetracylines, chloramphenicols, and streptomycins; including within this group the following compounds: gramicidin, neomycin, polymyxin beta sulfate, tetracycline, benzethonium chloride, gentamicin sulfate, nitrofurazone, benzalkonium chloride, hexylresorcinol, chloroxylenol, cloflucarban, carbolic acid (phenol), triclocarban, triclosan, and mixtures thereof.

For compositions that contain an anesthetic, examples of anesthetics that can be used include, but are not limited to, benzocaine, lidocaine, lidocaine hydrochloride, dibucaine, dibucaine hydrochloride, procaine, tetracaine, tetracaine hydrochloride, tronothane, dyclonine, dyclonine hydrochloride, pramoxine hydrochloride, benzyl alcohol, diperodon, butamben picrate, cyclomethycaine sulfate, dimethisoquin hydrochloride, and mixtures thereof.

A pharmaceutical composition in accordance with the present invention can be used to treat and/or prevent:
a. cheilitis, inflammation of the lips
b. prevent and treat fever blisters (herpes infections)
c. prevent and treat bacterial infections [including Methicillin-resistant Staphylococcus aureus (MRSA)]
d. treat pruritus (itching)
e. treat and prevent cold sores
f. prevent secondary infection.

Compositions in accordance with the present invention can be used to treat and/or prevent mouth lesions, including:
a. cold sores
b. prevent and treat fever blisters (herpes infections)
c. prevent and treat bacterial infections (including MRSA)
d. treat pruritus (itching)
e. treat and prevent cold sores
f. prevent secondary infection.

As used herein, the term “treat” means to reduce the extent of inflammation or infection associated with a lip condition. Depending on the context, the term “treat” can also mean to prevent infection or inflammation when the pharmaceutical composition is used as a preventative agent. A “therapeutically effective amount” is an amount sufficient to reduce the extent of inflammation or infection, or to prevent infection or inflammation. The dose and treatment regimen will depend upon the individual characteristics of the subject being treated and the properties of the particular pharmaceutical composition, and can be determined in consultation with the subject's physician. Although a subject could be an animal, the subject is preferably a person with a lip condition.

In use, a pharmaceutical composition of the present invention can be applied to the affected lip one or more times daily. For example, patients treated for cheilitis will have less inflammation of the lips following application of the lip balm twice daily. This is especially important for those being treated with oral isotretinoin. Cheilitis can also be caused by irritation due to sun damage, irritants, or contact irritants. The invention provides a flavorful, hypoallergenic, all-natural balm with steroidal, sunscreen, antiviral, antibiotic, and/or bacteriostatic properties for treatment and/or prevention of skin disease, infections, and various other diseases, and to promote a better quality of life.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular configurations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized according to the present invention. Accordingly, the appended claims are intended to include within their scope such processes, machines, manufacture, compositions of matter, means, methods, or steps.

What is claimed is:

1. A pharmaceutical composition for treating a lip condition, comprising:
a) a therapeutically effective amount of a corticosteroid, or a physiologically acceptable salt thereof, and
b) a physiologically acceptable carrier,
with the proviso that the corticosteroid does not include hydrocortisone.

2. The pharmaceutical composition of claim 1, wherein the corticosteroid is selected from the group consisting of triamcinolone acetonide, mometasone, hydrocortisone acetate, betamethasone valerate, fluocinolone acetonide, flupamesone, dexamethasone, hydrocortisone acetate, hydroxy hydrocortisone, hydrocortisone valerate, triamcinolone hexacetonide, amcinonide, fluocinolone acetonide, fluocinonide, flurandrenolide, diflurrasone diacetate, betamethasone dipropionate, betamethasone, betamethasone benzoate, betamethasone valerate, halcinonide, desoximethasone, desonide, prednisolone, clocortolone pivalate, physiologically acceptable salts thereof, and mixtures thereof.

3. The pharmaceutical composition of claim 2, wherein the corticosteroid is triamcinolone acteonide.

4. The pharmaceutical composition of claim 1, further comprising a suncreen active, an antiviral agent, an antifungal agent, an antibacterial agent, or an anesthetic, or any combination thereof.

5. The pharmaceutical composition of claim 1, wherein the composition is in the form of a lip balm.

6. The pharmaceutical composition of claim 5, wherein the corticosteroid of the lip balm is triamcinolone acteonide.

7. The pharmaceutical composition of claim 1, wherein the lip condition is inflammation or infection.

8. The pharmaceutical composition of claim 7, wherein the lip condition is cheilitis.

9. A pharmaceutical composition for treating a lip condition, comprising triamcinolone acteonide, silica gel micronized, wax, petrolatum, potassium sorbate, butylated hydrotoluene, stevia, and mineral oil.

10. A method of treating a lip condition in a subject in need of such treatment, comprising applying a pharmaceutical composition to one or both lips of the subject under conditions sufficient to treat the lip condition,
wherein the pharmaceutical composition comprises:
a) a therapeutically effective amount of a corticosteroid, or a physiologically acceptable salt thereof, and
b) a physiologically acceptable carrier,
with the proviso that the corticosteroid does not include hydrocortisone.

11. The method of claim 10, wherein the lip condition is inflammation or infection.

12. The method of claim 11, wherein the lip condition is cheilitis.

13. The method of claim 10, wherein the corticosteroid is selected from the group consisting of triamcinolone acetonide, mometasone, hydrocortisone acetate, betamethasone valerate, fluocinolone acetonide, flupamesone, dexamethasone, hydrocortisone acetate, hydroxy hydrocortisone, hydrocortisone valerate, triamcinolone hexacetonide, amcinonide, fluocinolone acetonide, fluocinonide, flurandrenolide, diflurrasone diacetate, betamethasone dipropionate, betamethasone, betamethasone benzoate, betamethasone valerate, halcinonide, desoximethasone, desonide, prednisolone, clocortolone pivalate, physiologically acceptable salts thereof, and mixtures thereof.

14. The method of claim 13, wherein the corticosteroid is triamcinolone acteonide.

15. The method of claim 14, wherein the lip condition is cheilitis.
